(19) United States
(12) Patent Application Publication
(10) Pub. No.: US 2019/0387962 A1
(43) Pub. Date: Dec. 26, 2019

(54) ENDOSCOPE INSERTION SHAPE OBSERVATION APPARATUS AND DISPLAY METHOD FOR ENDOSCOPE INSERTION SHAPE OBSERVATION APPARATUS

(71) Applicant: OLYMPUS CORPORATION, Tokyo (JP)

(72) Inventors: Takechiyo NAKAMITSU, Hachioji-shi (JP); Kensuke MIYAKE, Hamura-shi (JP); Akira MURATA, Inagi-shi (JP); Isamu NAKAJIMA, Sagamiahara-shi (JP)

(73) Assignee: OLYMPUS CORPORATION, Tokyo (JP)

(21) Appl. No.: 16/514,356

(22) Filed: Jul. 17, 2019

(30) Foreign Application Priority Data
Jan. 17, 2017 (JP) 2017-006046

(51) Int. Cl. A61B 1/045; A61B 1/00; A61B 1/005

(52) U.S. Cl. CPC A61B 1/045; A61B 1/00006; G02B 23/2407; A61B 1/00045; A61B 1/00009; A61B 1/0051

(57) ABSTRACT

An endoscope insertion shape observation apparatus has a processor configured to detect an insertion shape of an insertion portion inserted into a subject P, and calculate operation information, including one of easy insertion information or difficult insertion information, of insertion operation obtained in operation of inserting the insertion portion. The operation information is determined based on an insertion length of the insertion portion. The processor is also configured to simultaneously display of a result of the generation and the insertion shape on the monitor, and change between the easy insertion information and the difficult insertion information.

### CROSS REFERENCE TO RELATED APPLICATION

This application is a continuation application of PCT/JP2017/034793 filed on Sep. 26, 2017 and claims benefit of Japanese Application No. 2017-006046 filed in Japan on Jan. 17, 2017, the entire contents of which are incorporated herein by this reference.

### BACKGROUND

Exemplary embodiments disclose an endoscope insertion shape observation apparatus configured to observe an endoscope insertion state and a display method for endoscope insertion shape observation apparatus.

Endoscope apparatuses comprising an endoscope configured to pick up an image of an object inside a subject, a video processor configured to generate an observation image of the object picked up by the endoscope, and a monitor configured to display the observation image generated by the video processor have been widely used in a medical field, in an industrial field and in other fields. However, it is unknown from observation images how an insertion portion of an endoscope is inserted into a subject.

Therefore, in order to make it possible to see the insertion state of an endoscope when the endoscope is inserted, endoscope insertion shape observation apparatuses have been developed by providing a plurality of source coils incorporated into an insertion portion, a receiving antenna composed of a plurality of sense coils arranged in coil blocks, and a monitor configured to display an insertion shape of the insertion portion.

When an insertion portion of an endoscope is inserted into a subject, the insertion portion of the endo scope may take a shape such as a loop and a stick shape that prevents insertion of the insertion portion of the endoscope. An operator confirms an insertion shape of the insertion portion displayed on a monitor and then performs operation to release such a state.

For example, Japanese Patent Application Laid-Open Publication No. 2007-54401 discloses an endoscope insertion shape analysis apparatus configured to display an insertion shape of an endoscope, and display information for assisting operation to release situations including, for example, a loop and a stick state.

### SUMMARY

An endoscope insertion shape observation apparatus can include a processor configured to detect an insertion shape of an insertion portion inserted into a subject, calculate operation information of insertion operation obtained in operation of inserting the insertion portion, generate information to display on a first display apparatus, the information being appropriate information in a part leading to easy insertion operation of the insertion portion or appropriate information in a part leading to difficult insertion operation according to an insertion length of the insertion portion, and control simultaneous display of a result of the generation and the insertion shape on the first display apparatus and control a change between the appropriate information in a part leading to easy insertion operation and the appropriate information in a part leading to difficult insertion operation to display on the first display apparatus according to the insertion length of the insertion portion.

Additionally, one aspect of a display method for endoscope insertion shape observation apparatus according to the present invention comprises steps of detecting an insertion shape or an insertion position of an insertion portion inserted into a subject, selecting operation information for operator by referring to setting information for operator according to the insertion shape or the insertion position, selecting operation information for supervisory doctor by referring to setting information for supervisory doctor according to the insertion shape or the insertion position, and simultaneously displaying an image of the subject generated by an endoscope image generating apparatus, the insertion shape of the insertion portion, the operation information for operator and the operation information for supervisory doctor on an display apparatus for operator.

Further, another aspect of a display method for endoscope insertion shape observation apparatus according to the present invention comprises steps of detecting an insertion shape or an insertion position of an insertion portion inserted into a subject, selecting operation information for supervisory doctor by referring to setting information for supervisory doctor according to the insertion shape or the insertion position, and simultaneously displaying an image of the subject generated by an endoscope image generating apparatus, an operator operation image picked up by an image pickup apparatus, the operation information for supervisory doctor and the insertion shape of the insertion portion on a display apparatus for supervisory doctor.

### BRIEF DESCRIPTION OF THE DRAWINGS

FIG. 1 is a block diagram showing an endoscope insertion shape observation apparatus according to an exemplary embodiment;

FIG. 2 is a configuration diagram showing an entire configuration of a medical system including the endoscope insertion shape observation apparatus shown in FIG. 1;

FIG. 3 is an explanatory diagram to explain how to use the endoscope insertion shape observation apparatus;

FIG. 4 is block diagram showing an example of a specific configuration of a probe 21;

FIG. 5 is a diagram showing an example of setting information set in an operation information setting section 41 according to an exemplary embodiment;

FIG. 6A is an explanatory diagram showing a display image displayed on a display screen of a monitor 50 before and after reaching a specific part;

FIG. 6B is an explanatory diagram showing a display image displayed on the display screen of the monitor 50 when there is a specific shape;

FIG. 7 is a flowchart to explain how an exemplary embodiment works;

FIG. 8 is a diagram showing an example of setting information set in the operation information setting section 41 according to a modification;

FIG. 9A is an explanatory diagram showing an example of a display image displayed on the display screen of the monitor 50 according to the modification;

FIG. 9B is an explanatory diagram showing an example of a display image displayed on the display screen of the monitor 50 according to the modification;

FIG. 9C is an explanatory diagram showing an example of a display image displayed on the display screen of the monitor 50 according to the modification;

FIG. 9D is an explanatory diagram showing an example of a display image displayed on the display screen of the monitor 50 according to the modification;

FIG. 9E is an explanatory diagram showing an example of a display image displayed on the display screen of the monitor 50 according to the modification;

FIG. 10 is a flowchart to explain how the modification works;

FIG. 11 is a block diagram showing an endoscope insertion shape observation apparatus according to an exemplary embodiment;

FIG. 12 is an explanatory diagram showing a display image displayed on the display screen of the monitor 50 after elapse of a predetermined time period;

FIG. 13 is a flowchart to explain the process of an exemplary embodiment

FIG. 14A is an explanatory diagram showing a display image displayed on the display screen of the monitor 50;

FIG. 14B is an explanatory diagram showing a display image displayed on the display screen of the monitor 50;

FIG. 14C is an explanatory diagram showing a display image displayed on the display screen of the monitor 50;

FIG. 15A is an explanatory diagram showing a display image displayed on the display screen of the monitor 50 according to a specific part through which the insertion portion 4b is passing;

FIG. 15B is an explanatory diagram showing a display image displayed on the display screen of the monitor 50 according to a specific part through which the insertion portion 4b is passing;

FIG. 15C is an explanatory diagram showing a display image displayed on the display screen of the monitor 50 according to a specific part through which the insertion portion 4b is passing;

FIG. 16A is an explanatory diagram showing a display image displayed on the display screen of the monitor 50 during the insertion portion 4b passing through the sigmoid colon;

FIG. 16B is an explanatory diagram showing a display image displayed on the display screen of the monitor 50 during the insertion portion 4b passing through the sigmoid colon;

FIG. 16C is an explanatory diagram showing a display image displayed on the display screen of the monitor 50 after the insertion portion 4b passing through the sigmoid colon;

FIG. 17 is a block diagram showing an endoscope insertion shape observation apparatus according to an exemplary embodment;

FIG. 18A is an explanatory diagram showing a display image for operator displayed on the display screen of the monitor 50;

FIG. 18B is an explanatory diagram showing a display image for supervisory doctor displayed on a display screen of a monitor 110;

FIG. 19 is a diagram showing an example of size information set according to an insertion length or a specific shape;

FIG. 20A is an explanatory diagram showing a display image displayed on the display screen of the monitor 110 during the insertion portion 4b passing through the rectum;

FIG. 20B is an explanatory diagram showing a display image displayed on the display screen of the monitor 110 during the insertion portion 4b passing through the sigmoid colon;

FIG. 20C is an explanatory diagram showing a display image displayed on the display screen of the monitor 110 when there is a specific shape (loop);

FIG. 21A is an explanatory diagram showing a display image displayed on the display screen of the monitor 110 during the insertion portion 4b passing through the rectum; and

FIG. 21B is an explanatory diagram showing a display image displayed on the display screen of the monitor 110 during the insertion portion 4b passing through the sigmoid colon.

### DETAILED DESCRIPTION

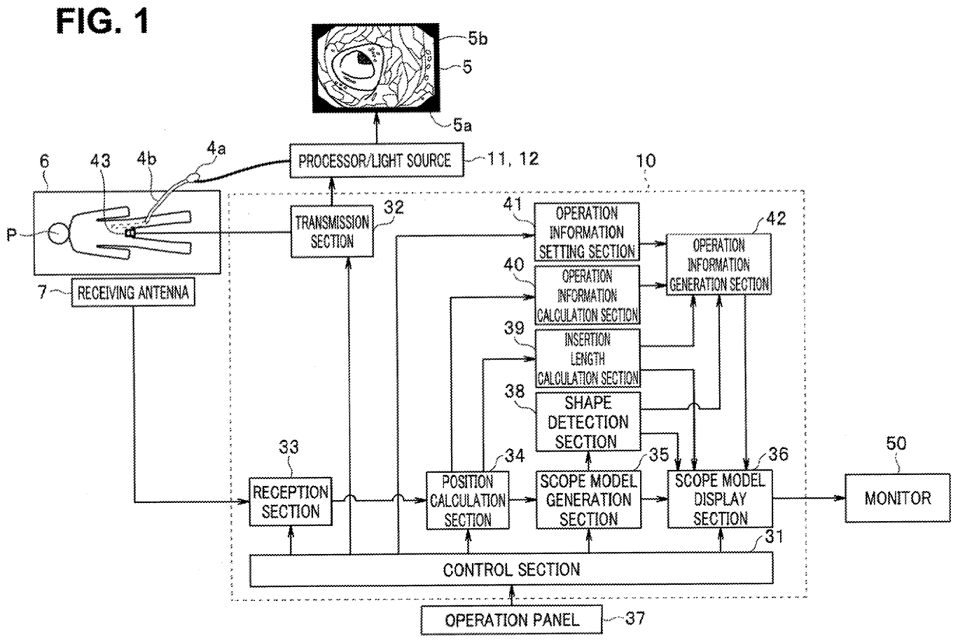

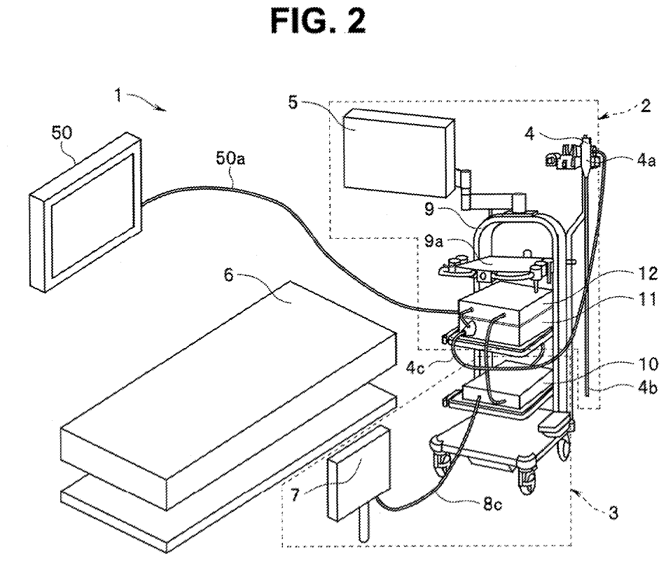

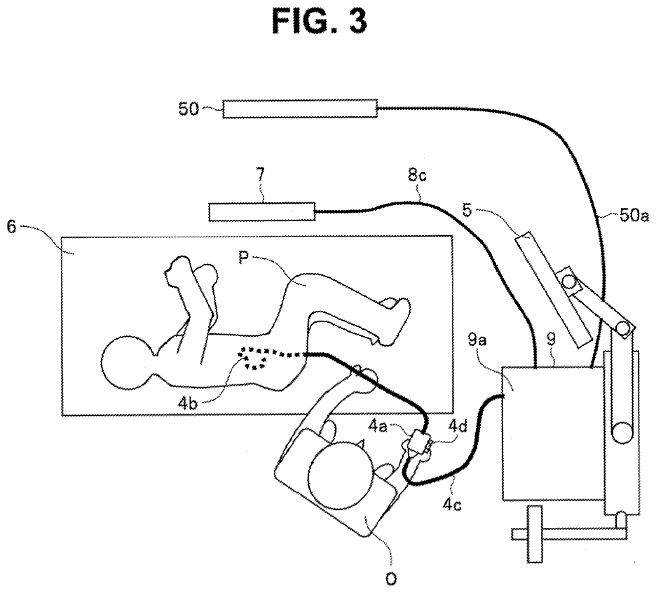

FIG. 1 is a block diagram showing an endoscope insertion shape observation apparatus according to an exemplary embodiment. Additionally, FIG. 2 is a configuration diagram showing an entire configuration of a medical system including the endoscope insertion shape observation apparatus shown in FIG. 1. Further, FIG. 3 is an explanatory diagram to explain how to use the endoscope insertion shape observation apparatus.

The apparatus can arbitrarily change operation information to display according to an insertion position of an insertion portion of an endoscope, whereby providing optimum information for operator according to the insertion position.

In FIG. 2 and FIG. 3, a medical system 1 is configured to include an endoscope apparatus 2 and an endoscope insertion shape observation apparatus 3. The endoscope apparatus 2 includes an endoscope 4, a light source apparatus 11, a processor 12 and a monitor 5. The endoscope 4 has an elongated and flexible insertion portion 4b inserted into a body cavity of a subject P as a subject, an operation section 4a that is connected to a base end of the insertion portion 4b and provided with various actuators, and a cable 4c for connecting the operation section 4a and the processor 12.

FIG. 2 shows an example in which the light source apparatus 11 and the processor 12 are placed on a medical trolley 9. The monitor 5 is also attached to a movable arm provided in the medical trolley 9. The endoscope 4 can be hung on a hook of the medical trolley 9.

FIG. 3 shows a state in which the insertion portion 4b is inserted from the anus into the large intestine of the subject P lying on a bed 6 for inspection. FIG. 3 shows a state of an operator O who holds the operation section 4a of the endoscope 4 that are connected to the processor 12 on the medical trolley 9 by the cable 4c, and the insertion portion 4b.

The light source apparatus 11 generates illumination light for illuminating the subject. Illumination light from the light source apparatus 11 is guided to a distal end portion of the insertion portion 4b through a light guide inserted into the insertion portion 4b of the endoscope 4 so that the subject is irradiated with light from the distal end portion of the insertion portion 4b. An image pickup device not shown is arranged at the distal end portion of the insertion portion 4b. The image pickup device has a light receiving surface to form an optical image of an object by reflected light (return light) received from and reflected by the subject. The image pickup device is driven and controlled by the processor 12 so as to convert an optical image of an object into an image signal that is outputted to the processor 12. The processor 12 serving as an endoscope image generating apparatus has an image signal processing section not shown. The image signal processing section receives an image signal from the image pickup device and processes the signal, followed by outputting an endoscope image obtained after signal processing to the monitor 5. Thus, an endoscope image 5b of the subject is displayed on a screen 5a of the monitor 5.

A bending section is provided at a tip of the insertion portion 4b. The bending section is bent and driven by a bending knob 4d provided in the operation section 4a. An operator can push the insertion portion 4b into a body cavity while bending the bending section by operating the bending knob 4d.

In an exemplary embodiment, the endoscope insertion shape observation apparatus 3 can be configured to observe an insertion state of the insertion portion 4b includes a control unit 10, a probe 21 for insertion state detection, a receiving antenna 7 and a monitor 50. The control unit 10 of the endoscope insertion shape observation apparatus 3 is placed on the medical trolley 9 and the probe 21 for insertion state detection is inserted into the insertion portion 4b as described later. The receiving antenna 7 is connected to the control unit 10 by a cable 8c.

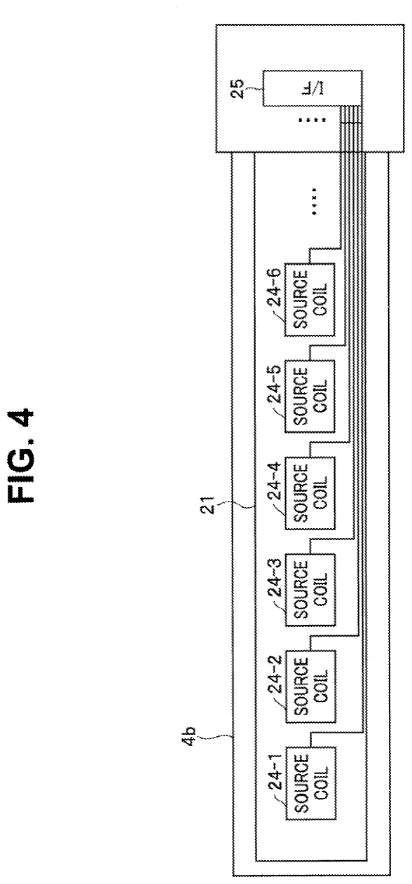

FIG. 4 is a block diagram showing an example of a specific configuration of the probe 21. As shown in FIG. 4, the probe 21 is inserted into a treatment instrument insertion channel not shown inside the insertion portion 4b. In the probe 21, a plurality of transmission coils 24-1, 24-2, . . . (hereinafter, simply referred to as transmission coils 24 unless each of the transmission coils needs to be distinguished) are installed along an axis of the probe at, for example, predetermined intervals. The probe 21 is inserted into the treatment instrument insertion channel and a tip or rear end of the probe 21 is fixed, whereby the plurality of transmission coils 24-1, 24-2, . . . are arranged in an axial direction of the insertion portion 4b at predetermined intervals.

The probe 21 can be fixedly inserted into the treatment instrument insertion channel of the endoscope 4 in order to incorporate the transmission coils 24 into the insertion portion 4b of the endoscope 4, though the transmission coils 24 may also be directly inserted into the insertion portion 4b of the endoscope 4.

The receiving antenna 7 has a plurality of coil blocks not shown and is arranged, for example, on a side of the bed 6. Each of the coil blocks of the receiving antenna 7 is composed of, for example, three sense coils wound in three directions so that respective coil surfaces are disposed orthogonally. In the receiving antenna 7 as a whole, for example, four coil blocks or twelve sense coils are arranged. Each of the sense coils is configured to detect a signal proportional to the intensity of a magnetic field of an axial direction component orthogonal to a coil surface of the sense coil. For example, the coil blocks are configured to receive a generated magnetic field and convert the magnetic field into a voltage signal which is outputted as a detection result. Operational states of the probe 21 and the receiving antenna 7 are controlled by the control unit 10.

As shown in FIG. 1, the control unit 10 is provided with a control section 31 as a processor including hardware. Entire or partial functions of a plurality of circuits of the processor may be executed by software. For example, the processor including hardware may have a central processing unit (CPU), ROM and RAM and various programs stored in ROM and corresponding to various functions may be read and executed by the CPU. The control section 31 controls the control unit 10 as a whole. Note that a memory not shown stores not only a program describing processing of the control section 31 but also data or the like for use in positional calculation to be described later.

The control section 31 controls a transmission section 32. The transmission section 32 is composed of, for example, FPGA and controlled by the control section 31 to generate and output, for example, a sine wave signal to drive the probe 21 by using waveform data from which a magnetic field is generated in the probe 21. Note that the transmission section 32 is controlled by the control section 31 so as to supply a sine wave to each of the coils 24 of the probe 21 individually. In other words, the control section 31 can control which transmission coils 24 of the probe 21 has a supply of a sine wave.

Each of the transmission coils 24 has a supply of a high-frequency sine wave from the control unit 10 via an I/F 25. A high-frequency sine wave is applied to each of the transmission coils 24 that can therefore radiate an electromagnetic wave involving a magnetic field over the surroundings. Note that the control unit 10 can drive the respective transmission coils 24-1, 24-2, . . . sequentially at appropriate time intervals or, for example, at intervals of several m seconds. The control unit 10 can also separately specify timing at which each of the transmission coils 24-1, 24-2, . . . generates a magnetic field.

The receiving antenna 7 uses the sense coils to receive magnetic fields generated by the transmission coils 24 and convert the magnetic fields into voltage signals that are outputted as detection results. The detection results from the receiving antenna 7 are fed to a reception section 33 of the control unit 10. The reception section 33 applies predetermined signal processing such as amplification processing to a signal received from the receiving antenna 7, followed by outputting the signal thus processed to a position calculation section 34.

The position calculation section 34 is composed of, for example, DSP and processes frequency extraction (Fourier transform: FT) to inputted digital data to separate and extract magnetic field detection information of a frequency component corresponding to a high-frequency sine wave of each of the transmission coils 24. Then, each digital data of the separated magnetic field detection information is used to calculate spatial position coordinates of each of the transmission coils 24 provided in the probe 21. A calculation result of the position coordinates by the position calculation section 34 is supplied to a scope model generation section 35, an insertion length calculation section 39 and an operation information calculation section 40. The scope model generation section 35 serving as an insertion shape detection section generates a line image by connecting position coordinates of each of the transmission coils 24 as an insertion shape image. The insertion shape image generated by the scope model generation section 35 is fed to a scope model display section 36 and a shape detection section 38.

The shape detection section 38 can detect, based on an insertion shape image sent from the scope model generation section 35, a predetermined shape of the insertion portion 4b in the body cavity. For example, shape patterns indicating a linear shape, a stick shape, a loop shape, a deflection shape and other shapes are stored in the shape detection section 38 so that whether an insertion shape image forms any of the shape patterns can be detected, whereby making it possible to detect whether a shape of the insertion portion 4b falls in any of a linear shape, a stick shape, a loop shape, a deflection shape or other shapes. The shape detection section 38 is configured to output information of a detected shape to the scope model display section 36 and an operation information generation section 42.

The insertion length calculation section 39 calculates a length of the insertion portion 4b inserted into the body cavity. A portion of the insertion portion 4b in which any of the transmission coils 24 having position coordinates detected as corresponding to position coordinates of the anus by the position calculation section 34 is arranged is determined to be located in the anus so that the insertion portion 4b is inserted into the body cavity from the position of the coil 24 having position coordinates detected as corresponding to position coordinates of the anus to the tip of the insertion portion 4b. A position of each of the transmission coils 24 inserted into the insertion portion 4b from the tip of the insertion portion 4b is already known. The insertion length calculation section 39 calculates an insertion length from a position of any of the coils 24 located in the anus position to the tip of the insertion portion 4b. The insertion length calculation section 39 is configured to output information of a calculated insertion length (e.g. insertion length provided in the unit of 1 cm) to the scope model display section 36 and the operation information generation section 42.

In order to set a position of the anus of the subject P, for example, a marker 43 is employed. The marker 43 incorporates a transmission coil not shown and a high-frequency sine wave sent from the transmission section 32 is applied to the transmission coil. When a high-frequency sine wave sent from the transmission section 32 is applied, the marker 43 generates a magnetic field. This magnetic field is received by the receiving antenna 7 and a detection result of the receiving antenna 7 is supplied to the position calculation section 34 via the reception section 33. Therefore, the position calculation section 34 can obtain position coordinates of the marker 43 in a measuring coordinate system.

The operation information calculation section 40 calculates operation information of insertion operation obtained when insertion operation is performed by the insertion portion 4b. The operation information calculation section 40 calculates the operation information of insertion operation from position coordinates calculated by the position calculation section 34. The operation information calculated by the operation information calculation section 40 includes eight items that are “upper/lower angle direction,” “left/right angle direction,” “left-hand operation direction (twist) & operation amount,” “right-hand operation direction (twist) & operation amount,” “tip speed,” “tip direction vector,” “maximum curvature” and “depth.” Note that the “left-hand operation direction (twist) & operation amount” and the “right-hand operation direction (twist) & operation amount” are obtained by visualizing hand twist operation that is important to advance the endoscope and may also be expressed by, for example, a direction and a size of an angular velocity (rad/s) of the transmission coils 24 calculated from position coordinates of the position calculation section 34. Additionally, it is not necessary to limit the operation to twist operation and, for example, a direction and a size of hand push/pull operation may also be displayed by using a direction and a size of a moving amount of the transmission coils 24 calculated from position coordinates of the position calculation section 34. Note that the operation information calculated by the operation information calculation section 40 is not limited to the above eight items. The operation information calculation section 40 is configured to output the calculated operation information to the operation information generation section 42.

In an operation information setting section 41, the operation information to display on the monitor 50 according to an insertion position of the insertion portion 4b is associated with the insertion position of the insertion portion 4b and set.

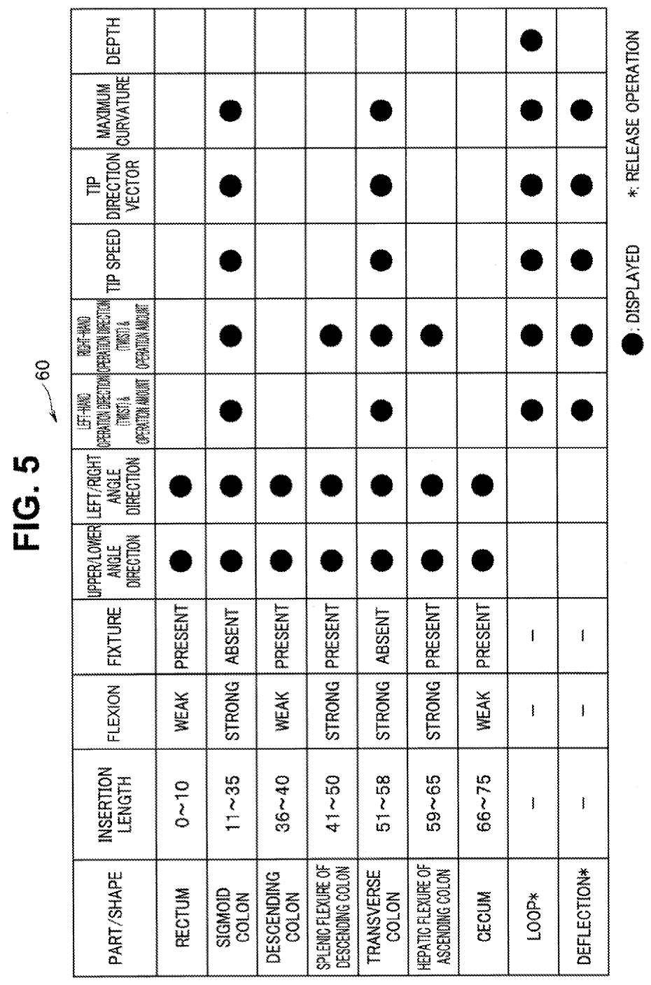

FIG. 5 is a diagram showing one example of setting information set in the operation information setting section 41 according to an exemplary embodiment.

To examine the large intestine, the insertion portion is inserted in an order from the anus to the rectum, the sigmoid colon, the descending colon, the splenic flexure of the descending colon, the transverse colon, the hepatic flexure of the ascending colon and the cecum. Therefore, parts included in the setting information 60 shown in FIG. 5 are the rectum, the sigmoid colon, the descending colon, the splenic flexure of the descending colon, the transverse colon, the hepatic flexure of the ascending colon and the cecum. In the setting information 60, the respective parts are associated with insertion lengths and the operation information to display.

The insertion length of the insertion portion can be helpful for determining which part of the large intestine the insertion portion is located. The insertion difficulty of the insertion portion is different depending on what part of the large intestine the insertion portion is inserted into. For example, the rectum is a portion of the large intestine that does not bend or curve, and thus, weak flexion of the insertion portion is used. The sigmoid colon is a portion of the large intestine that does include a bend or curve and thus, strong flexion of the insertion portion is used. FIG. 5 indicates various portions of the large intestine including the rectum, the sigmoid colon, the descending colon, the splenic flexure of the descending colon, the transverse colon, the hepatic flexure of the ascending colon and the cecum. In FIG. 5 at the column labeled FLEXION, an indication of whether weak or strong flexion of the insertion portion is required at each part of the large intestine.

When the insertion portion 4b has a specific shape such as a loop shape and a deflection shape, the specific shape needs to be released to make the insertion portion 4b return to a neutral state (a state without having a specific shape) for a moment. Therefore, the setting information 60 is associated with the operation information to display a specific shape of the insertion portion 4b.

The setting information 60 of the operation information setting section 41 is set by the control section 31 that received an operation signal from an operation panel 37. The operation panel 37 can receive user operation by an operator and output the operation signal based on the user operation to the control section 31. Through the operation panel 37, an operator can set any operation information to display according to a specific part or a specific shape. The operation information setting section 41 is configured to output the setting information 60 thus set to the operation information generation section 42.

The operation information generation section 42 generates the operation information to display on the monitor 50 according to an insertion length calculated by the insertion length calculation section 39 and the setting information 60 received from the operation information setting section 41, and outputs the operation information to the scope model display section 36. For example, when an insertion length calculated by the insertion length calculation section 39 is 2 cm, the operation information generation section 42 refers to the setting information 60 and determines that a part into which the insertion portion 4b is inserted is the rectum. Then, the operation information generation section 42 refers to the setting information 60 and generates two items of the operation information or information of the “upper/lower angle direction” and the “left/right angle direction” that are outputted to the scope model display section 36.

Additionally, when a shape of the insertion portion 4b detected by the shape detection section 38 is a specific shape (a loop shape or a deflection shape), the operation information generation section 42 generates six items of the operation information or information of the “left-hand operation direction (twist) & operation amount,” the “right-hand operation direction (twist) & operation amount,” the “tip speed,” the “tip direction vector,” the “maximum curvature” and the “depth” that are outputted to the scope model display section 36.

Thus, the insertion shape image generated by the scope model generation section 35, the information of the specific shape detected by the shape detection section 38, the information of the insertion length calculated by the insertion length calculation section 39, and the operation information generated by the operation information generation section 42 are fed to the scope model display section 36.

The scope model display section 36 serving as a first control section makes a control to simultaneously display an insertion shape image generated by the scope model generation section 35 and the operation information generated by the operation information generation section 42 on the monitor 50 (first display apparatus). The scope model display section 36 also makes a control to display the information of the insertion length calculated by the insertion length calculation section 39 or the information of the specific shape detected by the shape detection section 38 simultaneously with the insertion shape image and the operation information.

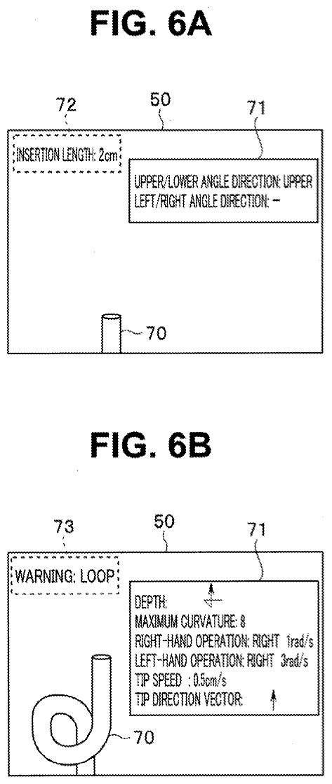

Here, explanation of display images displayed on the monitor 50 by the scope model display section 36 is given. FIG. 6A is an explanatory diagram showing a display image displayed on the display screen of the monitor 50 before and after reaching a specific part, and FIG. 6B is an explanatory diagram showing a display image displayed on the display screen of the monitor 50 when there is a specific shape.

First, FIG. 6A is used to explain the display image obtained before or after reaching a specific part. Here, explanation of a case where an insertion length calculated by the insertion length calculation section 39 is 2 cm is given.

The scope model display section 36 receives information of an insertion shape 70 generated by the scope model generation section 35.

The operation information generation section 42 also refers to the setting information 60 based on the information of the insertion length calculated by the insertion length calculation section 39, and determines the operation information to display. When an insertion length is 2 cm, for example, the operation information generation section 42 determines to display the “upper/lower angle direction” and the “left/right angle direction” from the setting information 60. The operation information generation section 42 generates, from the operation information calculated by the operation information calculation section 40, the operation information of the “upper/lower angle direction” and the “left/right angle direction” that are outputted to the scope model display section 36. In other words, the scope model display section 36 receives operation information 71 shown in FIG. 6A.

The scope model display section 36 further receives information of an insertion length 72 calculated by the insertion length calculation section 39.

The scope model display section 36 generates a display image to simultaneously display inputted information of the insertion shape 70, the operation information 71 and the insertion length 72 and outputs the display image to the monitor 50, whereby making a control to display the display image shown in FIG. 6A on the monitor 50. Note that even though the scope model display section 36 simultaneously displays three kinds of information relating to the insertion shape 70, the operation information 71 and the insertion length 72, it is not limited to this and, for example, two kinds of information relating to the insertion shape 70 and the operation information 71 may also be displayed simultaneously on the monitor 50.

Next, FIG. 6B is used to explain a display image when a specific shape is detected. Here, explanation of a case where a shape detected by the shape detection section 38 is a loop shape is given.

The scope model display section 36 receives information of the insertion shape 70 generated by the scope model generation section 35.

Also, the operation information generation section 42 refers to the setting information 60 based on information of a shape detected by the shape detection section 38, and determines the operation information to display. For example, when a shape detected by the shape detection section 38 is a loop shape, the operation information generation section 42 generates from the setting information 60 the operation information of the “left-hand operation direction (twist) & operation amount,” the “right-hand operation direction (twist) & operation amount,” the “tip speed,” the “tip direction vector,” the “maximum curvature” and the “depth” that are outputted to the scope model display section 36. In other words, the scope model display section 36 receives the operation information 71 shown in FIG. 6B.

The scope model display section 36 further receives information of a specific shape 73 detected by the shape detection section 38.

The scope model display section 36 generates a display image to simultaneously display inputted information of the insertion shape 70, the operation information 71 and the specific shape 73 and outputs the display image to the monitor 50, whereby making a control to display the display image shown in FIG. 6B on the monitor 50.

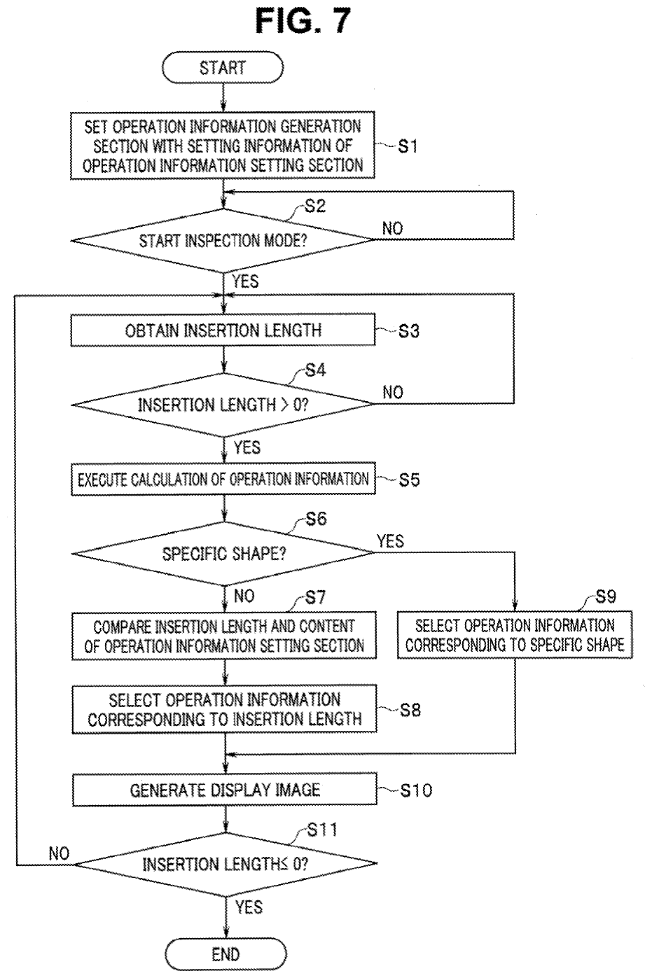

FIG. 7 is a flowchart to explain how an exemplary embodiment works.

First, the operation information generation section 42 is set with the setting information 60 of the operation information setting section 41 (S1) and it is determined whether an inspection mode is started (S2). The setting information 60 set in the operation information setting section 41 is inputted to the operation information generation section 42. When the control section 31 determines that the inspection mode is not started (S2: NO), the process returns to S2 to repeat the same processing. In contrast, when the control section 31 determines that the inspection mode is started (S2: YES), the process moves onto S3.

Next, the insertion length calculation section 39 obtains an insertion length (S3) and it is determined whether the insertion length is greater than 0 or not (S4). Information of the insertion length obtained by the insertion length calculation section 39 is inputted to the scope model display section 36 and the operation information generation section 42. When it is determined that the insertion length is not greater than 0 (S4: NO), the process returns to S3 to repeat the same processing. In contrast, when it is determined that the insertion length is greater than 0 (S4: YES), the operation information calculation section 40 calculates the operation information (S5). The operation information thus calculated is inputted to the scope model display section 36 and the operation information generation section 42.

Next, information of a shape detected by the shape detection section 38 is inputted to the operation information generation section 42. The operation information generation section 42 determines whether the shape detected by the shape detection section 38 is a specific shape or not (S6).

When the operation information generation section 42 determines that the shape detected by the shape detection section 38 is not the specific shape (S6: NO), the operation information generation section 42 compares the insertion length with a content of the setting information 60 (S7) and selects the operation information corresponding to the insertion length (S8). The operation information generation section 42 refers to the setting information 60 to select the operation information (the operation information 71 shown in FIG. 6A) corresponding to the insertion length. In other words, the operation information generation section 42 refers to the setting information 60 shown in FIG. 5 to select the optimum operation information corresponding to the insertion length.

For example, when the insertion length falls in a range of 0 to 10 cm, a part of the insertion portion 4b that has a weak flexion is in the rectum and is fixed to the abdomen. This results in an easy insertion operation, in which two items of the operation information are generated according to contents set in the setting information 60. In contrast, when the insertion length falls in a range of 11 to 35 cm, a part of the insertion portion 4b that has a strong flexion is inserted into the sigmoid colon in a dangling state and is not fixed to the abdomen. This results in a difficult insertion operation, in which seven items of the operation information are generated corresponding to contents set in the setting information 60. The operation information selected according to an insertion length is inputted to the scope model display section 36.

On the other hand, when the operation information generation section 42 determines that the shape detected by the shape detection section 38 is the specific shape (S6: YES), the operation information generation section 42 selects the setting information corresponding to the specific shape (S9). The operation information generation section 42 refers to the setting information 60 to select the operation information (operation information shown in FIG. 6B) corresponding to the specific shape. The operation information selected corresponding to the specific shape is inputted to the scope model display section 36.

Then, the scope model display section 36 generates a display image from the inputted information to display on the monitor 50 (S10). Then, the control section 31 determines whether an insertion length is equal to 0 or less (S11). When the control section 31 determines that the insertion length is not equal to 0 or less (S11: NO), the process returns to S3 to repeat the same processing. In contrast, when the control section 31 determines that the insertion length is equal to 0 or less (S11: YES), the processing is finished.

Note that the scope model display section 36 may also be configured to generate a display image to display on the monitor 50 only when the insertion portion 4b is inserted into the large intestine and not to generate a display image while the insertion portion 4b is pulled out of the large intestine. In other words, the scope model display section 36 generates the display image shown in FIG. 6A or FIG. 6B only during inspection in which the insertion portion 4b is inserted into the large intestine. Then, the scope model display section 36 may be configured so as not to generate the display image shown in FIG. 6A or FIG. 6B when the inspection is finished and the insertion portion 4b is pulled out of the large intestine. For example, the scope model display section 36 determines that it is during inspection when an insertion length is on the increase for a predetermined time period, and determines that inspection is finished when an insertion length is on the decrease for a certain time period.

As stated above, in the present embodiment, the endoscope insertion shape observation apparatus 3 changes the operation information to generate, according to the setting information 60 set in the operation information setting section 41 and the insertion length calculated by the insertion length calculation section 39. As a result, the endoscope insertion shape observation apparatus 3 can change the operation information to display on the monitor 50 according to a part into which the insertion portion 4b is inserted and which leads to easy insertion operation, and a part which leads to difficult insertion operation.

Hence, according to the endoscope insertion shape observation apparatus of the present embodiment, the optimum operation information can be displayed according to the insertion position of the endoscope insertion portion.

Additionally, in the present embodiment, the endoscope insertion shape observation apparatus 3 is configured to change the operation information to generate, according to the setting information 60 set in the operation information setting section 41 and the specific shape detected by the shape detection section 38. As a result, the endoscope insertion shape observation apparatus 3 can display, on the monitor 50, the operation information required in release operation to release the specific shape when the shape of the insertion portion 4b corresponds to the specific shape which makes insertion difficult.

The present embodiment aims at providing information (operation information) that leads to improvement of insertion by operators based on an insertion shape, in which information that is not needed by supervisory doctors is displayed or information that is needed by supervisory doctors may not be displayed. Therefore, for example, when a supervisory doctor supervises a medical intern or a less-experienced doctor, it takes time for the supervisory doctor to search necessary information and comprehend contents or the supervisory doctor may not be able to obtain necessary information.

Therefore, in a modification, explanation is given with regard to an endoscope insertion shape observation apparatus that is capable of displaying only operation information required by supervisory doctors depending on an insertion position.

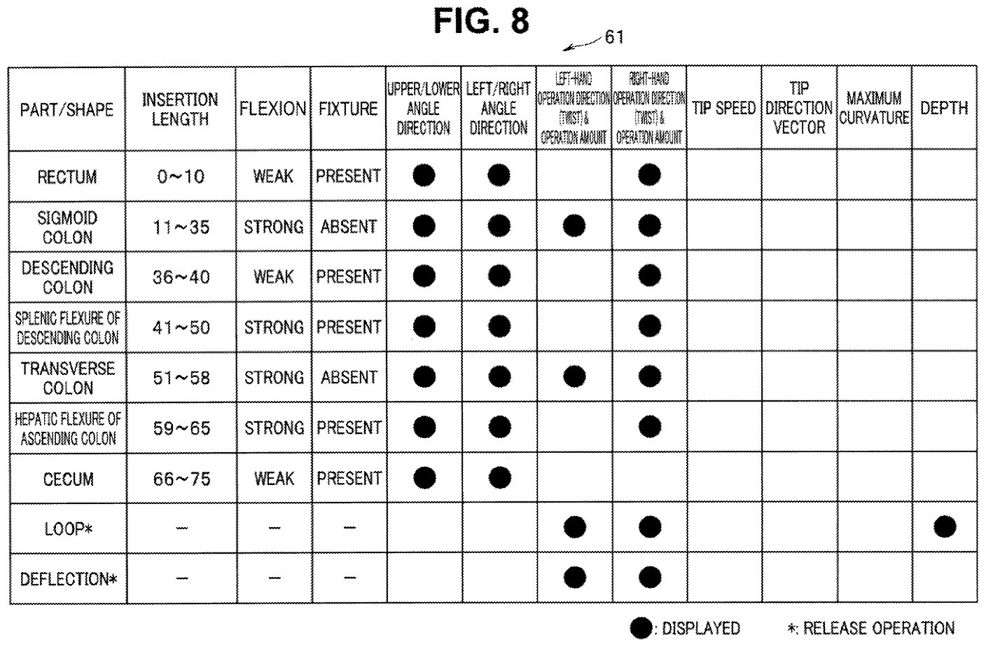

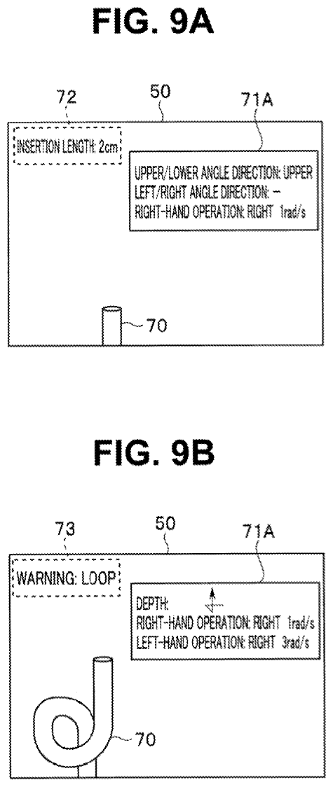

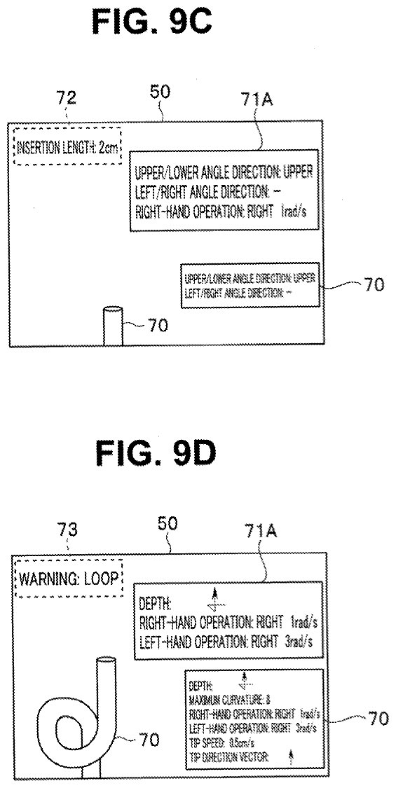

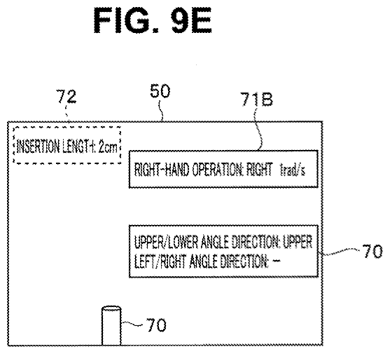

The endoscope insertion shape observation apparatus according to the modification has the same configuration as a whole as the configuration of the endoscope insertion shape observation apparatus 3 according to the previously described embodiment. In the modification, operation information set in the operation information setting section 41 is different from the operation information in the previously described embodiment. FIG. 8 is a diagram showing one example of setting information set in the operation information setting section 41 according to the modification. Additionally, FIG. 9A to FIG. 9E are explanatory diagrams showing examples of display images displayed on the display screen of the monitor 50 according to modification.

A supervisory doctor uses the operation panel 37 to set items of the operation information to display before and after the insertion portion 4b reaches a specific part and when a specific shape is formed. The operation information is set in the operation information setting section 41 through a control made by the control section 31. Therefore, setting information 61 for supervisory doctor as shown in FIG. 8 is set in the operation information setting section 41. For example, when a mode is changed to a supervisory doctor display mode by the operation panel 37, the setting information 61 for supervisory doctor which is set in the operation information setting section 41 is inputted to the operation information generation section 42.

The operation information generation section 42 outputs corresponding operation information 71A according to the setting information 61 for supervisory doctor and an insertion length to the scope model display section 36A to display on the monitor 50 as shown in FIG. 9A. Alternatively, the operation information generation section 42 generates the corresponding operation information 71A according to the setting information 61 for supervisory doctor and the specific shape and outputs the corresponding operation information to the scope model display section 36 to display the corresponding operation information on the monitor 50 as shown in FIG. 9B.

Note that in addition to the setting information 61 for supervisory doctor, the setting information 60 for operator as shown in FIG. 5 may also be set in the operation information setting section 41 so that the setting information 60 for operator and the setting information 61 for supervisory doctor are inputted to the operation information generation section 42. Owing to this input, the operation information 71 generated from the setting information 60 for operator and the operation information 71A generated from the setting information 61 for supervisory doctor may be displayed simultaneously on a monitor 50b as shown in FIG. 9C and FIG. 9D so that an operator and a supervisory doctor can individually confirm these pieces of operation information. Alternatively, display contents of the operation information 71A may be displayed larger than or in a different color from display contents of the operation information 71 so that the operation information 71 and the operation information 71A are displayed according to different display methods. A display method may also be determined depending on a result obtained from comparing information contents between the operation information 71 and the operation information 71A. For example, when the operation information 71 and the operation information 71A have a small amount of information as shown in FIG. 9C, operation information 71B obtained from a difference of contents between the setting information 60 and the setting information 61 may be displayed as shown in FIG. 9E to allow effective use of a display area.

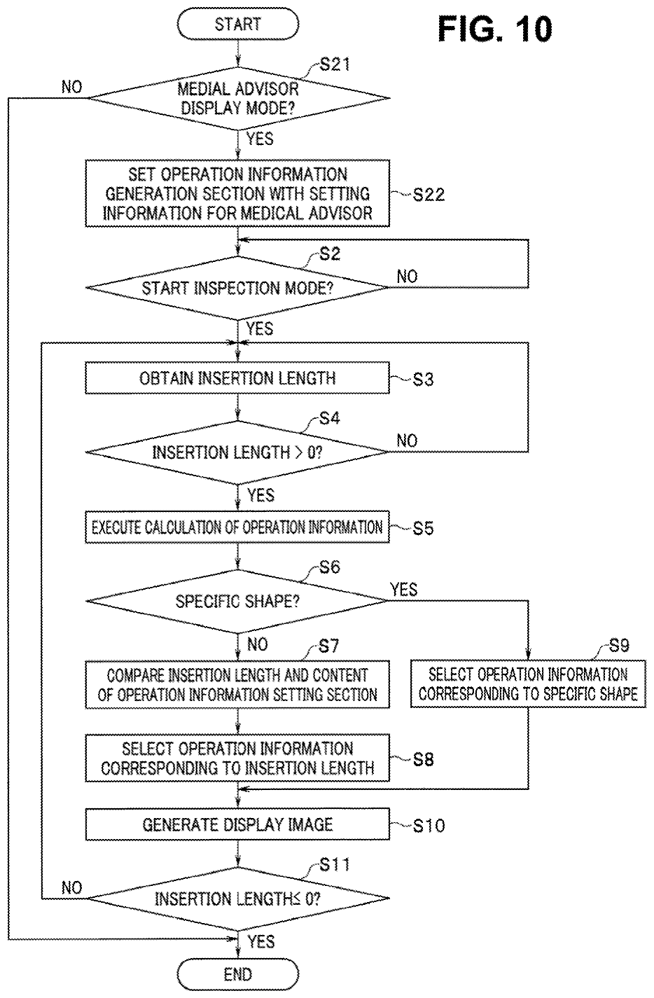

Next, explanation as to how the modification thus configured works is given with reference to FIG. 10. FIG. 10 is a flowchart to explain how the modification works. Note that the processes shown in FIG. 7 are provided with the same reference characters in FIG. 10 and explanation of the same processes is omitted.

First, the control section 31 determines whether it is a supervisory doctor display mode or not (S21). When the control section 31 determines that it is not the supervisory doctor display mode (S21: NO), the process is finished. In contrast, when the control section 31 determines that it is the supervisory doctor display mode (S21: YES), the control section 31 sets the operation information generation section 42 with the setting information 61 for supervisory doctor from the operation information setting section 41 (S22).

Then, when an inspection mode starts at S3, the operation information to display is selected according to the setting information 61 for supervisory doctor and the insertion length or according to the setting information 61 for supervisory doctor and the specific shape in the same manner as previously described. Then, a display image including the selected operation information is generated and displayed on the monitor 50.

As stated above, in the present modification, the endoscope insertion shape observation apparatus 3 can select the operation information required by supervisory doctors according to the specific part or the specific shape to display on the monitor 50. Therefore, supervisory doctors can reduce the time spent for searching the operation information or comprehension of contents so that changes of overlooking of display contents is also minimized. As a result, supervisory doctors can more appropriately point out things to medical interns in a more immediate manner than before.

Display of the operation information 71 displayed on the monitor 50 is switched sequentially as time passes. This may cause, for example, supervisory doctors to overlook necessary operation information. Hence, an endoscope insertion shape observation apparatus configured to simultaneously display real-time operation information and past operation information is explained in the present embodiment. Note that the present embodiment provides explanation of control made in a supervisory doctor mode though it is not limited to the supervisory doctor mode.

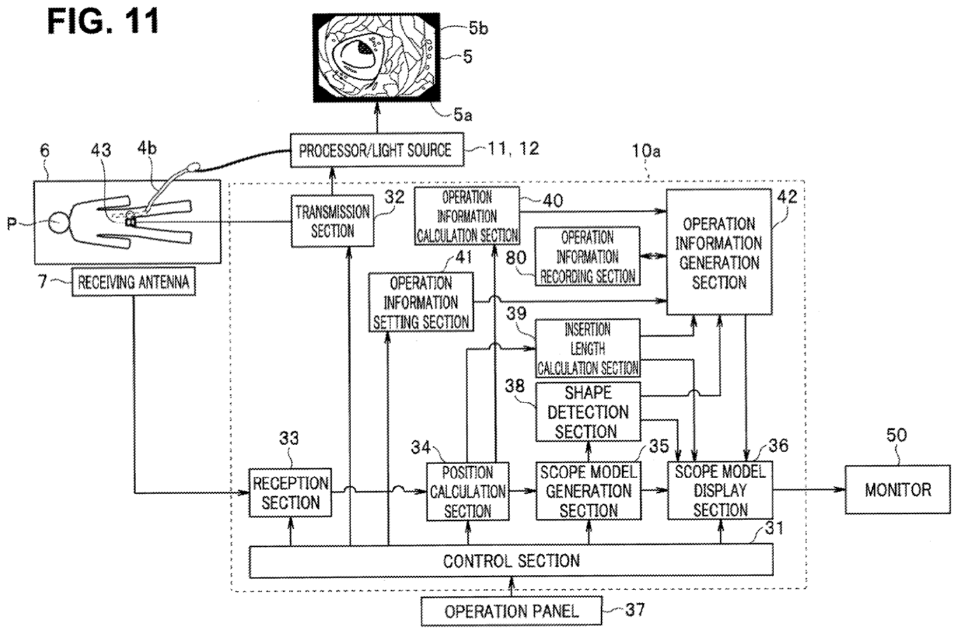

FIG. 11 is a block diagram showing the endoscope insertion shape observation apparatus according to the present embodiment. Note that components shown in FIG. 1 are provided with the same reference characters in FIG. 11 and explanation of the same components is omitted.

An endoscope insertion shape observation apparatus 3 according to the present embodiment is configured to have a control unit 10a in which an operation information recording section 80 is added to the control unit 10 shown in FIG. 1.

The operation information generation section 42 associates generated real-time operation information with current time to record in the operation information recording section 80. The operation information generation section 42 generates, after elapse of a predetermined time period, the real-time operation information and the operation information (past operation information) obtained before the predetermined time period that are outputted to the scope model display section 36. Any time periods can be set as the predetermined time period by a user who is, for example, a supervisory doctor with the use of the operation panel 37. The control section 31 sets the predetermined time period according to an operation signal received from the operation panel 37.

For example, when the predetermined time period is set to 3 seconds, the operation information generation section 42 reads the operation information recorded three seconds before in the operation information recording section 80 and outputs the generated real-time operation information with the operation information recorded three seconds before and read from the operation information recording section 80 to the scope model display section 36.

The scope model display section 36 generates a display image to simultaneously display the real-time operation information, the past operation information, the above insertion shape and the information of the insertion length, and outputs the display image to the monitor 50.

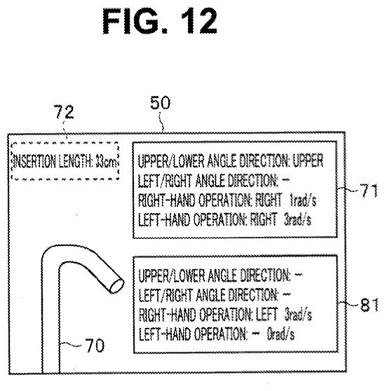

FIG. 12 is an explanatory diagram showing a display image displayed on the display screen of the monitor 50 after elapse of a predetermined time period. Note that components shown in FIG. 6A are provided with the same reference characters in FIG. 12.

The operation information generation section 42 refers to the setting information 61 based on the information of the insertion length calculated by the insertion length calculation section 39 and determines the operation information to display. For example, when an insertion length is 33 cm, a specific part is determined to be the sigmoid colon and the operation information generation section 42 generates the operation information 71 (real-time operation information) including the “upper/lower angle direction,” the “left/right angle direction,” the “left-hand operation direction (twist) & operation amount” and the “right-hand operation direction (twist) & operation amount.” The operation information generation section 42 also reads the operation information 81 recorded before a predetermined time period from the operation information recording section 80. The operation information generation section 42 generates the generated real-time operation information 71 and the past operation information 81 read from the operation information recording section 80 that are outputted to the scope model display section 36.

The scope model display section 36 further receives the information of the insertion shape 70 generated by the scope model generation section 35 and the information of the insertion length 72 calculated by the insertion length calculation section 39.

The scope model display section 36 generates a display image to simultaneously display the insertion shape 70 thus inputted, the real-time operation information 71, the past operation information 81 and the information of the insertion length 72, and outputs the display image to the monitor 50, thereby making a control to display a display image shown in FIG. 12 on the monitor 50.

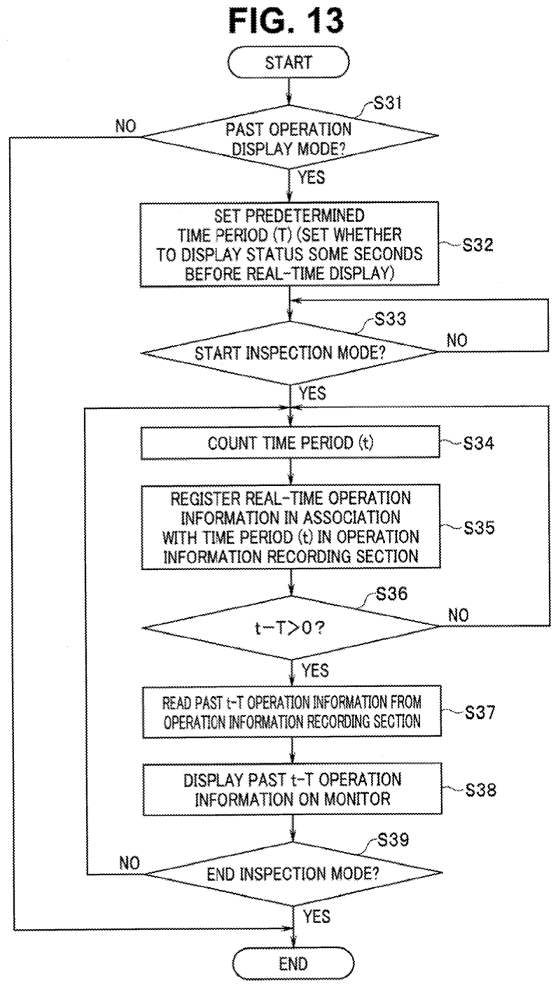

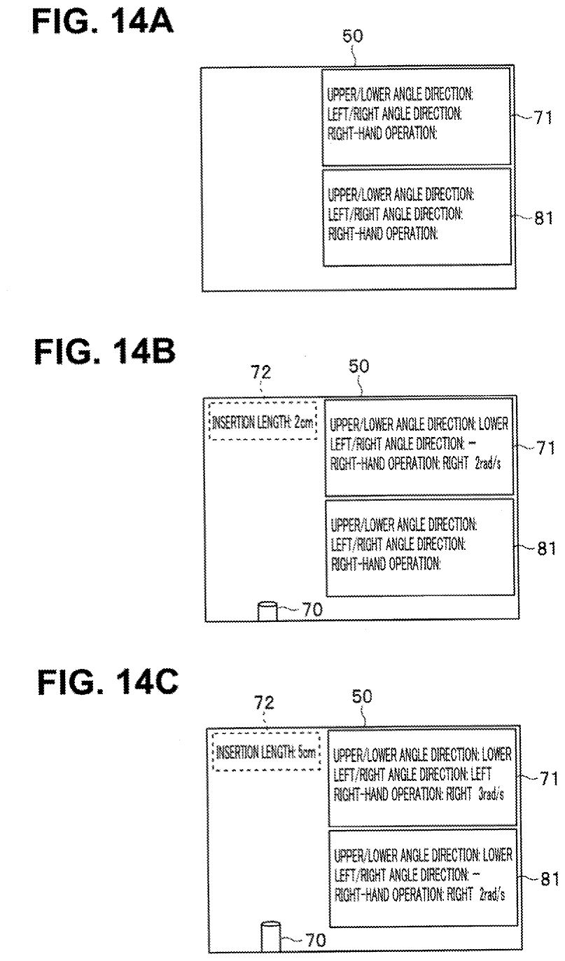

Next, how the embodiment thus configured works is explained with reference to FIG. 13, FIG. 14A, FIG. 14B and FIG. 14C. FIG. 14A, FIG. 14B and FIG. 14C are explanatory diagrams showing display images displayed on the display screen of the monitor 50.

First, the control section 31 determines whether it is a past operation display mode to display the past operation information 81 or not (S31). When the control section 31 determines that it is not the past operation display mode (S31: NO), the process is finished. In contrast, when the control section 31 determines that it is the past operation display mode (S31: YES), the control section 31 sets a predetermined time period (T) (S32). The predetermined time period (T) is used to set a status (operation information) obtained how many seconds before real-time display to display and any time periods can be set as the predetermined time period by using the operation panel 37.

Next, it is determined whether an inspection mode is started or not (S33). When the control section 31 determines that the inspection mode is not started (S33: NO), the process returns to S33 to repeat the same processing. In contrast, when the control section 31 determines that the inspection mode is started (S33: YES), the process moves onto S34.

The operation information generation section 42 counts a time period (t) (S4) and causes the operation information recording section 80 to record the time period (t) in association with the real-time operation information 71 (S35). For example, in a case of t=0, nothing is displayed in the real-time operation information and the past operation information 81 as shown in FIG. 14A.

Next, the operation information generation section 42 determines whether a value obtained by subtracting the predetermined time period (T) from the time period (t) is greater than 0 or not. When the operation information generation section 42 determines that a value obtained by subtracting the predetermined time period (T) from the time period (t) is not greater than 0 (S36: NO), the process returns to S34 to repeat the same processing. For example, when the predetermined time period (T) is set to three seconds and the time period (t) is one second, the process returns to S34 to continuously execute processing at S34 and S35. In this case, as shown in FIG. 14B, the monitor 50 displays data in the real-time operation information 71 and nothing in the past operation information 81.

In contrast, when the operation information generation section 42 determines that a value obtained by subtracting the predetermined time period (T) from the time period (t) is greater than 0 (S36: YES), the past operation information 81 corresponding to the value obtained by subtracting the predetermined time period (T) from the time period (t) is read from the operation information recording section 80 (S37). The past operation information 81 thus read is inputted along with the real-time operation information 71 to the scope model display section 36.

The scope model display section 36 displays, on the monitor, the past operation information 81 corresponding to the value obtained by subtracting the predetermined time period (T) from the time period (t) (S38). Thus, data in the real-time operation information 71 and the past operation information 81 are displayed simultaneously as shown in FIG. 14C.

Next, the control section 31 determines whether the inspection mode is finished or not (S39). When the control section 31 determines that the inspection mode is not finished (S39: NO), the process returns to 34 to repeat the same processing. In contrast, when the control section 31 determines that the inspection mode is finished (S39: YES), processing is finished.

As stated above, in the present embodiment, the endoscope insertion shape observation apparatus 3 is configured to simultaneously display the real-time operation information 71 and the past operation information 81 after elapse of a predetermined time period. As a result, supervisory doctors can confirm information that was overlooked due to display switching after the elapse of a predetermined time period.

The present embodiment is configured to constantly display the past operation information 81 on the monitor 50, though it is not limited to this. For example, the operation information generation section 42 may also be configured to switch display of the past operation information 81 to be turned ON or OFF according to a specific part through which the insertion portion 4b is passing.

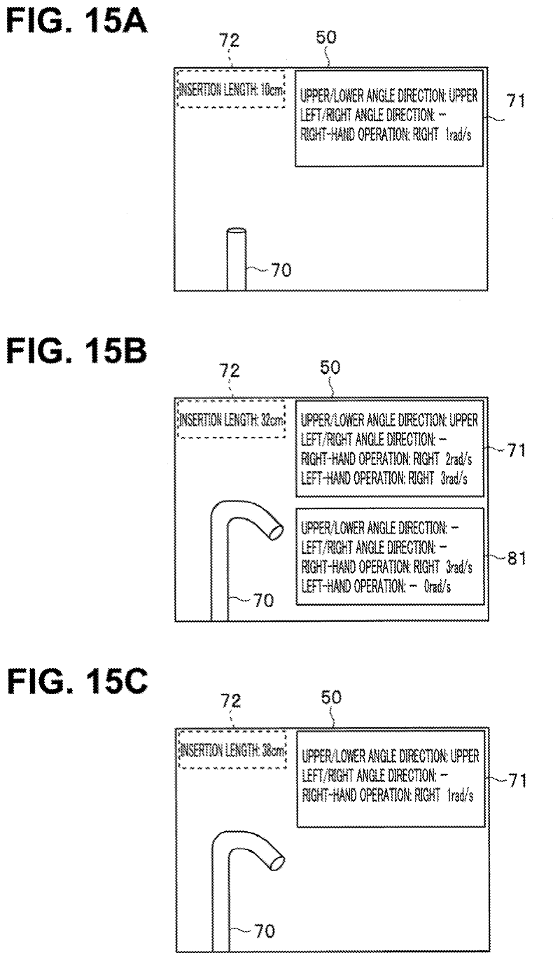

FIG. 15A, FIG. 15B and FIG. 15C are explanatory diagrams showing display images displayed on the display screen of the monitor 50 according to a specific part through which the insertion portion 4b is passing.

When the operation information generation section 42 determines that a part through which the insertion portion 4b is passing is the rectum according to the information of the insertion length, the operation information generation section 42 generates the real-time operation information 71 that is outputted to the scope model display section 36. This allows the monitor 50 to display the real-time operation information 71 as shown in FIG. 15A in addition to the information of the insertion shape 70 and the insertion length 72.

Additionally, when the operation information generation section 42 determines that a part through which the insertion portion 4b is passing is the sigmoid colon according to the information of the insertion length, the operation information generation section 42 generates the real-time operation information 71 and the past operation information 81 obtained a predetermined amount of seconds before that are outputted to the scope model display section 36. Thus, this allows the monitor 50 to display the real-time operation information 71 and the past operation information 81 as shown in FIG. 15B in addition to the information of the insertion shape 70 and the insertion length 72.

Additionally, when the operation information generation section 42 determines that a part through which the insertion portion 4b is passing is the descending colon according to the information of the insertion length, the operation information generation section 42 generates the real-time operation information 71 which is outputted to the scope model display section 36. This allows the monitor 50 to display the real-time operation information 71 as shown in FIG. 15C in addition to the information of the insertion shape 70 and the insertion length 72.

As such, when the specific part of insertion portion 4b is passing through the rectum or the descending colon leading to easy insertion operation, the scope model display section 36 makes a control to display only the real-time operation information 71 on the monitor 50. In contrast, when the specific part of the insertion portion 4b having a strong flexion is passing through the sigmoid colon leading to difficult insertion operation, the scope model display section 36 makes a control to display the real-time operation information 71 and the past operation information 81 on the monitor 50.

Additionally, the operation information generation section 42 may also cause the monitor 50 to collectively display the past operation information obtained in the insertion portion 4b passing through a specific part after the insertion portion 4b passed through the specific part.

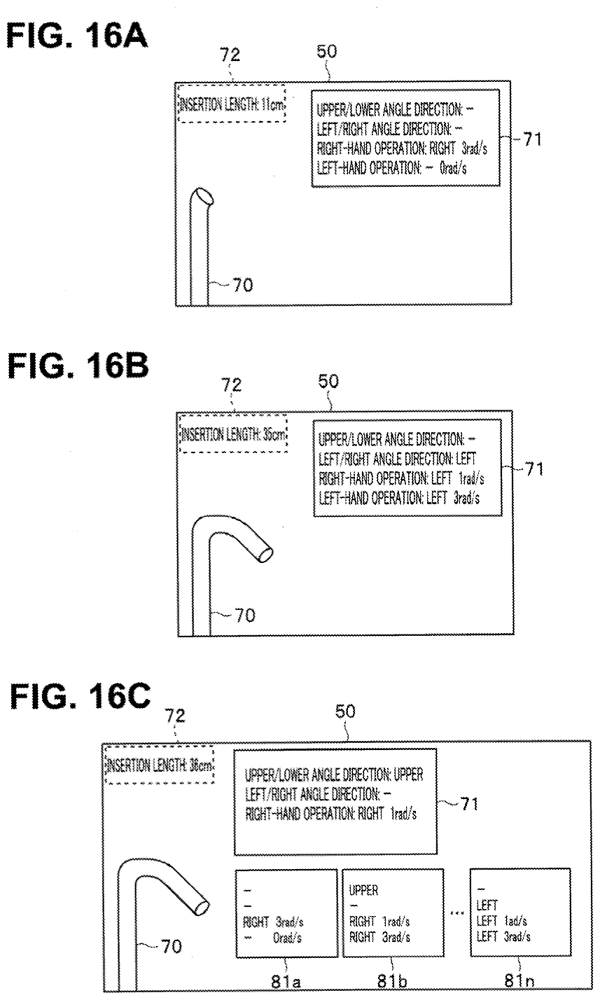

FIG. 16A and FIG. 16B are explanatory diagrams showing display images displayed on the display screen of the monitor 50 during the insertion portion 4b passing through the sigmoid colon. FIG. 16C is an explanatory diagram showing a display image displayed on the display screen of the monitor 50 after the insertion portion 4b passing through the sigmoid colon.

The operation information generation section 42 generates the real-time operation information 71 according to the information of the insertion length while the insertion portion 4b is passing through a specific part which is, for example, the sigmoid colon, and output the real-time operation information to the scope model display section 36. In other words, when an insertion length falls in a range of 11 cm to 35 cm, the scope model display section 36 makes a control to display the real-time operation information 71 in addition to the information of the insertion shape 70 and the insertion length 72 as shown in FIG. 16A and FIG. 16B.

Then, when the operation information generation section 42 determines that the insertion portion 4b passed through the sigmoid colon according to the information of the insertion length, the operation information generation section 42 reads all the past operation information obtained during the insertion portion 4b passing through the sigmoid colon, in addition to the real-time operation information 71, from the operation information recording section 80 and outputs these pieces of information to the scope model display section 36.

When the insertion length becomes 36 cm (while the insertion portion 4b is passing through the descending colon), the scope model display section 36 makes a control to collectively display, on the monitor 50, at least one or more past operation information 81a, 81b, . . . , 81n obtained during the insertion portion 4b passing through the sigmoid colon. As a result, in addition to the information of the insertion shape 70 and the insertion length 72 and the real-time operation information 71, all the past operation information 81a, 81b, . . . , 81n obtained during the insertion portion 4b passing through the sigmoid colon are displayed on the monitor 50 as shown in FIG. 16C.

Therefore, supervisory doctors can confirm a series of operations made by operators during the insertion portion 4b passing through a specific part on the monitor 50. For example, by confirming the series of operation, a supervisory doctor can confirm whether an operator is performing appropriate operation from entering a curvature part as a subject of inspection to passing through the curvature part.

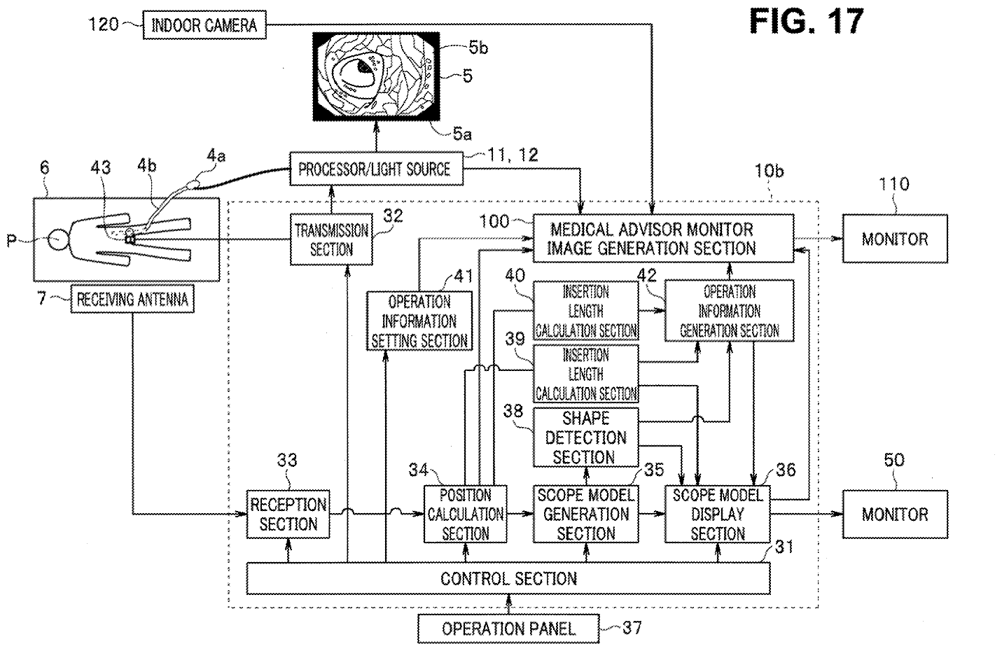

FIG. 17 is a block diagram showing an endoscope insertion shape observation apparatus according to an exemplary embodiment. Note that components shown in FIG. 1 are provided with the same reference characters in FIG. 17 and explanation of the same components is omitted.

An endoscope insertion shape observation apparatus 3 according to the present embodiment is configured by using a control unit 10b in place of the control unit 10 shown in FIG. 1. The control unit 10b is connected to the processor 12, a monitor 110 and an indoor camera 120. The control unit 10b is also configured to have a supervisory doctor monitor image generation section 100 added to the control unit 10 shown in FIG. 1.

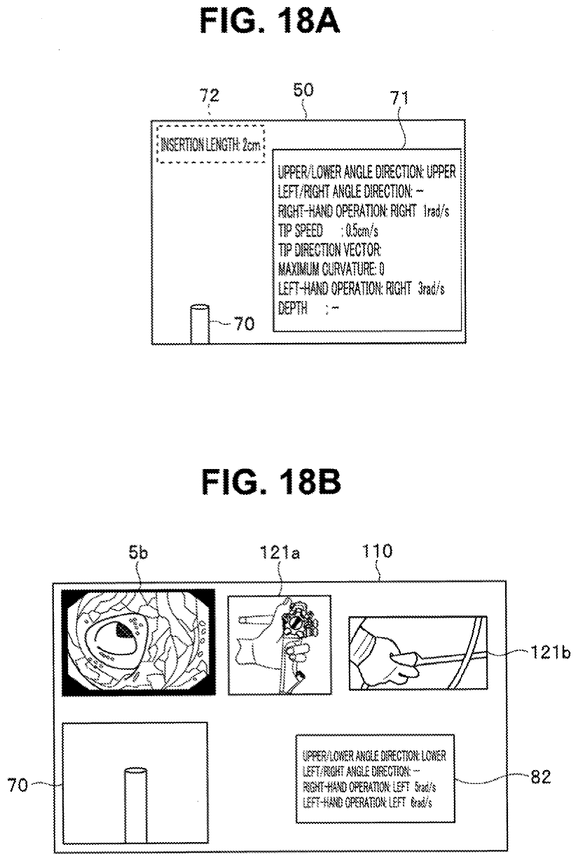

The supervisory doctor monitor image generation section 100 receives the endoscope image 5b of a subject which is generated by the processor 12. The indoor camera 120 also picks up an image of a hand of an operator (medical intern) or namely left-hand and right-hand operation images 121a, 121b of the operator (see FIG. 18B described below). The supervisory doctor monitor image generation section 100 receives the operation images of the operator picked up by the indoor camera 120. The supervisory doctor monitor image generation section 100 also receives the setting information 61 for supervisory doctor from the operation information setting section 41. The supervisory doctor monitor image generation section 100 further receives the operation information generated by the operation information generation section 42 and information of a display image generated by the scope model display section 36.

The supervisory doctor monitor image generation section 100 generates a display image for supervisory doctor based on each piece of the inputted information, and outputs the display image to the monitor 110. This allows the monitor 110 to display the display image for supervisory doctor.

As such, in the present embodiment, the monitor 50 serves as a monitor for an operator such as a medical intern and the monitor 110 serves as a monitor for a supervisory doctor. Then, a display image for an operator such as a medical intern is displayed on the monitor 50 and a display image for a supervisory doctor is displayed on the monitor 110. Here, explanation of display images displayed on the monitor 50 and the monitor 110 is given.

FIG. 18A is an explanatory diagram showing a display image for operator displayed on the display screen of the monitor 50, and FIG. 18B is an explanatory diagram showing a display image for supervisory doctor displayed on a display screen of the monitor 110.

The monitor 50 displays the information of the insertion shape 70, the operation information 71 and the insertion length 72 as shown in FIG. 18A in the same manner as previously described. However, in the present embodiment, the operation information generation section 42 does not receive the setting information 60 for operator (see FIG. 5) from the operation information setting section 41 as shown in FIG. 17. Therefore, the operation information generation section 42 generates eight pieces of the operation information 71 calculated by the operation information calculation section 40, and outputs the eight pieces of the operation information 71 to the scope model display section 36. Thus, a display image shown in FIG. 18A is generated by the scope model display section 36 and displayed on the monitor 50.

The monitor 110 displays, as shown in FIG. 18B, the endoscope image 5b of a subject, the left-hand operation image 121a of an operator, the right-hand operation image 121b of the operator, the insertion shape 70 and the operation information 82 for supervisory doctor. The endoscope image 5b of the subject is inputted from the processor 12 to the supervisory doctor monitor image generation section 100, and the left-hand and right-hand operation images 121a, 121b of the operator are inputted from the indoor camera 120 to the supervisory doctor monitor image generation section 100.

The insertion shape 70 is also included in information of a display image inputted from the scope model display section 36 to the supervisory doctor monitor image generation section 100.

Further, the eight pieces of the operation information 71 are inputted from the operation information generation section 42 to the supervisory doctor monitor image generation section 100. The supervisory doctor monitor image generation section 100 also receives the setting information 61 for supervisory doctor which is set by the operation information setting section 41. Information of the insertion length or the specific shape is also included in the information of the display image inputted from the scope model display section 36 to the supervisory doctor monitor image generation section 100.

The supervisory doctor monitor image generation section 100 refers to the setting information 61 according to the information of the insertion length or the specific shape and selects the operation information 82 for supervisory doctor to display on the monitor 110. In the example of FIG. 18B, a specific part is determined to be the sigmoid colon based on the insertion length and four pieces of information or the “upper/lower angle direction,” the “left/right angle direction,” the “left-hand operation direction (twist) & operation amount” and the “right-hand operation direction (twist) & operation amount” are selected as the operation information 82.

The supervisory doctor monitor image generation section 100 generates a display image in which the endoscope image 5b of the subject, the left-hand and right-hand operation images 121a, 121b of the operator, the insertion shape 70 and the operation information 82 for supervisory doctor are arranged as shown in FIG. 18B, and outputs the display image to the monitor 110. Note that arrangement of the endoscope image 5b of the subject, the left-hand and right-hand operation images 121a, 121b of the operator, the insertion shape 70 and the operation information 82 for supervisory doctor is not limited to the arrangement shown in FIG. 18B. Thus, the supervisory doctor monitor image generation section 100 serving as a second control section makes a control to simultaneously display the endoscope image 5b of the subject, the left-hand and right-hand operation images 121a, 121b of the operator, the insertion shape 70 and the operation information 82 for supervisory doctor on the monitor 110 (second display apparatus).

Conventionally, there have been cases where a supervisory doctor looks away from the monitor 50 displaying the operation information while watching the hands of an operator to confirm an operation state of the operator or while looking at the monitor 5 to confirm the endoscope image 5b.

In contrast, according to the present embodiment, the endoscope image 5b of the subject, the left-hand and right-hand operation images 121a, 121b of the operator, the insertion shape 70 and the operation information 82 for supervisory doctor are displayed on the monitor 110 for supervisory doctor. As a result, supervisory doctors do not need to look away from the operation information 82 in order to confirm an operation state of an operator or the endoscope image 5b and can therefore easily grasp entire operation of an operator. Further, supervisory doctors do not need to look away from the monitor 110 displaying the operation information 82 so that overlooking of the operation information 82 can be prevented.

Note that the supervisory doctor monitor image generation section 100 may also be configured to appropriately change a display size of the endoscope image 5b, the left-hand and right-hand operation images 121a, 121b of the operator, the insertion shape 70 and the operation information 82 for supervisory doctor according to the insertion length or the specific shape. Information of the display size is, for example, set in the operation information setting section 41 by using the operation panel 37 and through a control by the control section 31.

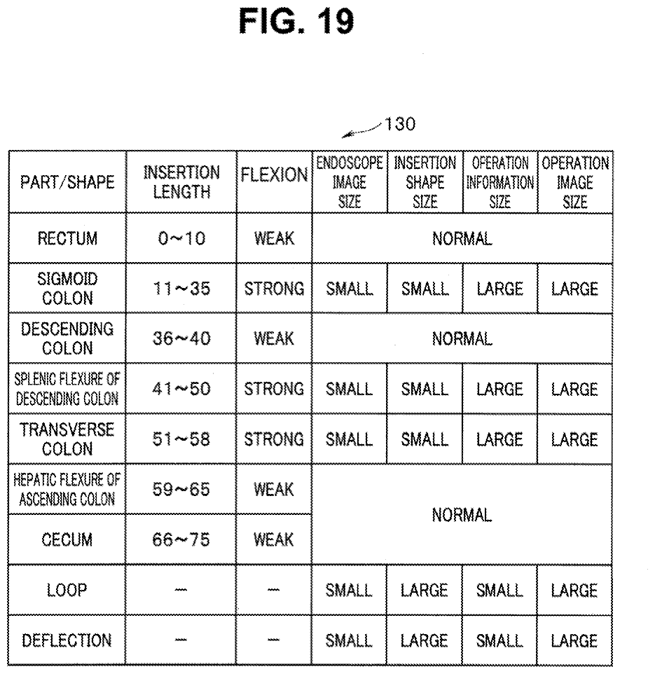

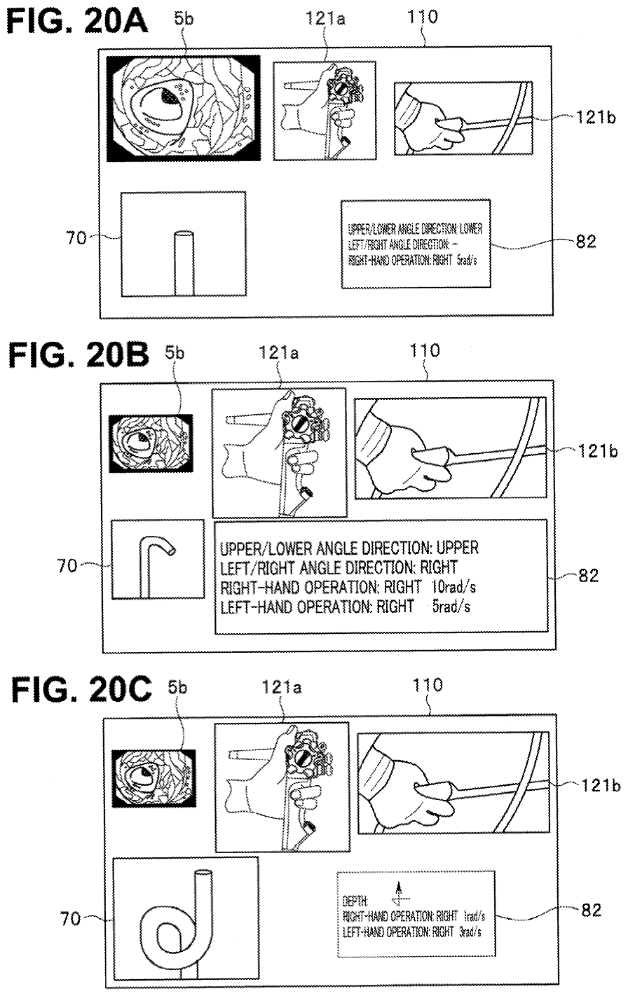

FIG. 19 is a diagram showing one example of size information set according to the insertion length or the specific shape. Additionally, FIG. 20A is an explanatory diagram showing a display image displayed on the display screen of the monitor 110 during the insertion portion 4b passing through the rectum, FIG. 20B is an explanatory diagram showing a display image displayed on the display screen of the monitor 110 during the insertion portion 4b passing through the sigmoid colon, and FIG. 20C is an explanatory diagram showing a display image displayed on the display screen of the monitor 110 when there is a specific shape (loop).

When the insertion portion 4b is passing through the rectum, both hands of an operator do not move vigorously so that a supervisory doctor needs to see all the information evenly. Therefore, as shown in the size information 130 of FIG. 19, the endoscope image 5b, the insertion shape 70, the operation information 82, the left-hand and right-hand operation images 121a, 121b of the operator are set to a standard size entirely.

The supervisory doctor monitor image generation section 100 makes a control to display, based on the size information 130, the endoscope image 5b, the insertion shape 70, the operation information 82, the left-hand and right-hand operation images 121a, 121b of the operator in a standard display size on the monitor 110 as shown in FIG. 20A.

Besides, when the insertion portion 4b is passing through the sigmoid colon, both hands of an operator move vigorously so that a supervisory doctor particularly needs to see the operation information 82 and the left-hand and right-hand operation images 121a, 121b of the operator. Therefore, as shown in the size information 130 of FIG. 19, the endoscope image 5b and the insertion shape 70 are set to a small size, whereas the operation information 82, and the left-hand and right-hand operation images 121a, 121b of the operator are set to a large size.

The supervisory doctor monitor image generation section 100 makes a control to display, based on the size information 130, the endoscope image 5b and the insertion shape 70 in a small display size and the operation information 82 and the left-hand and right-hand operation images 121a, 121b of the operator in a large size on the monitor 110 as shown in FIG. 20B.

In addition, when the insertion portion 4b forms a specific shape (loop), a supervisory doctor particularly needs to see the insertion shape 70, the left-hand and right-hand operation images 121a, 121b of the operator. Therefore, as shown in the size information 130 of FIG. 19, the endoscope image 5b and the operation information 82 are set to a small size, whereas the insertion shape 70 and the left-hand and right-hand operation images 121a, 121b of the operator are set to a large size.

The supervisory doctor monitor image generation section 100 makes a control to display, based on the size information 130, the endoscope image 5b and the operation information 82 in a small display size and the insertion shape 70 and the left-hand and right-hand operation images 121a, 121b of the operator in a large display size on the monitor 110 as shown in FIG. 20C.

Thus, the endoscope insertion shape observation apparatus 3 according to the present embodiment can enlarge and display information as needed by supervisory doctors according to the specific part through which the insertion portion 4b is passing or the specific shape of the insertion portion 4b, whereby making it easier for supervisory doctor to grasp necessary information.

Note that the present embodiment is configured to change display sizes of the endoscope image 5b, the insertion shape 70, the operation information 82 and the left-hand and right-hand operation images 121a, 121b of the operator according to the specific part or the specific shape, though the present embodiment is not limited to such a configuration and may also be configured to change display sizes according to, for example, the insertion shape 70 of the insertion portion 4b.

The present embodiment is also configured to have three display sizes of “small,” “standard” and “large” but the present embodiment is not limited to such sizes and may also have, for example, two or four sizes or more.

Furthermore, the present embodiment is also configured to display the endoscope image 5b, the insertion shape 70, the operation information 82 and the left-hand and right-hand operation images 121a, 121b of the operator on the monitor 110 for supervisory doctor, though information to display on the monitor 110 may also be changed according to, for example, the specific part or the specific shape. For example, during the insertion portion 4b passing through the rectum or during the insertion portion 4b passing through the sigmoid colon, information displayed on the display screen of the monitor 110 may be changed

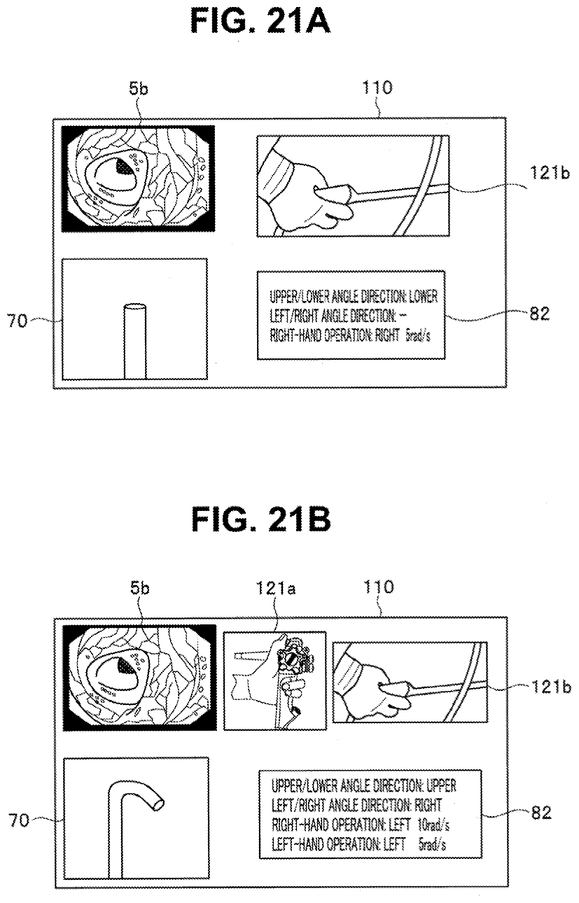

FIG. 21A is an explanatory diagram showing a display image displayed on the display screen of the monitor 110 during the insertion portion 4b passing through the rectum, and FIG. 21B is an explanatory diagram showing a display image displayed on the display screen of the monitor 110 during the insertion portion 4b passing through the sigmoid colon.

When the insertion portion 4b is passing through the rectum, operators perform operation of pushing the insertion portion 4b with the right hand but are less likely to use the left hand to operate the operation section 4a to bend the tip of the insertion portion 4b. Therefore, supervisory doctors do not need to confirm the left-hand operation image 121a of operators. Hence, the supervisory doctor monitor image generation section 100 makes a control to display the endoscope image 5b, the insertion shape 70, the operation information 82 and the right-hand operation image 121b of an operator on the monitor 110 as shown in FIG. 21A.

In contrast, when the insertion portion 4b is passing through the sigmoid colon, operators need to perform the operation of pushing (and operation of twisting) the insertion portion 4b with the right hand and to bend the tip of the insertion portion 4b by operating the operation section 4a with the left hand, in which it is important for the right-hand operation and the left-hand operation to cooperate with each other. Therefore, a supervisory doctor needs to see both left-hand and right-hand operation images 121a, 121b of an operator to confirm cooperation between the left-hand and right-hand operations by the operator. Hence, the supervisory doctor monitor image generation section 100 makes a control to display the endoscope image 5b, the insertion shape 70, the operation information 82 and the left-hand and right-hand operation images 121a, 121b of the operator on the monitor 110 as shown in FIG. 21B.

As a result, the endoscope insertion shape observation apparatus 3 can display only information that is needed by supervisory doctors on the monitor 110 according to the specific part through which the insertion portion 4b is passing or the specific shape of the insertion portion 4b, whereby making it easier for supervisory doctors to grasp necessary information.

Note that steps in the flowcharts of the present specification may be changed in terms of the execution order to execute several steps simultaneously or in different orders in each execution unless contrary to the nature of the steps.

The present invention is not limited to the aforementioned embodiments and various changes and modifications may be made within the scope of the present invention without changing the gist of the present invention.

What is claimed is:

1. An endoscope insertion shape observation apparatus comprising:
a processor configured to:
detect an insertion shape of an insertion portion of an endoscope configured to be inserted into a subject;
calculate a first operation information of insertion operation obtained in operation of inserting the insertion portion;
generate the operation information to display on a first display apparatus based on an insertion length of the insertion portion, the operation information being at least one of: easy insertion information for insertion of the insertion portion at an easy insertion area of the subject that does not bend or difficult insertion information for insertion of the insertion portion at a difficult insertion area of the subject that does bend;
simultaneously display the operation information and the insertion shape on the first display apparatus; and
change between the easy insertion information and the difficult insertion information as the insertion portion moves in the subject.

2. The endoscope insertion shape observation apparatus according to claim 1, wherein the operation information to display on the first display apparatus can be changed.

3. The endoscope insertion shape observation apparatus according to claim 1, wherein the processor is configured to simultaneously display the operation information comprising real-time operation information and past operation information generated a predetermined time period before the real-time operation information on the first display apparatus.

4. The endoscope insertion shape observation apparatus according to claim 3, wherein the processor is configured to control display or non-display of the past operation information on the first display apparatus.

5. The endoscope insertion shape observation apparatus according to claim 3, wherein the processor is configured to display one or more past operation information.

6. The endoscope insertion shape observation apparatus according to claim 1, wherein the processor is configured to simultaneously display an image of the subject generated by an endoscope image generating apparatus, an operation image for operator obtained by an image pickup apparatus, and the operation information on a second display apparatus.

7. The endoscope insertion shape observation apparatus according to claim 6, wherein the processor is configured to change sizes of the image of the subject, the operation image for operator, and the operation information to display on the second display apparatus.

8. A display method for endoscope insertion shape observation apparatus comprising steps of:
detecting an insertion shape or an insertion position of an insertion portion inserted into a subject;
selecting first operation information for operator by referring to setting information for operator according to the insertion shape or the insertion position;
selecting second operation information for supervisory doctor by referring to setting information for supervisory doctor according to the insertion shape or the insertion position; and
simultaneously displaying an image of the subject generated by an endoscope image generating apparatus, the insertion shape of the insertion portion, the first operation information for operator and the second operation information for supervisory doctor on a display apparatus for operator.

9. The display method for endoscope insertion shape observation apparatus according to claim 8, wherein some of the first operation information is different information from some of the second operation information, the display method further comprising displaying the different information on the display apparatus.

10. A display method for endoscope insertion shape observation apparatus comprising steps of:
detecting an insertion shape or an insertion position of an insertion portion of an endoscope inserted into a subject;
selecting operation information for supervisory doctor based on setting information for the insertion portion of the endoscope; and
simultaneously displaying an image of the subject generated by an endoscope image generating apparatus, an operator operation image picked up by an image pickup apparatus, the operation information for supervisory doctor, and the insertion shape of the insertion portion on a display apparatus.

11. The display method for endoscope insertion shape observation apparatus according to claim 10, wherein sizes of the image of the subject, the operation image for operator, the operation information for supervisory doctor and the insertion shape of the insertion portion are changed based on the insertion shape or the insertion position of the insertion portion of the endoscope.

12. The display method for endoscope insertion shape observation apparatus according to claim 11, wherein when the insertion portion passes through an easy insertion area of the subject that does not bend, the image of the subject, the operation image for operator, the operation information for supervisory doctor and the insertion shape of the insertion portion are set to a standard size to display on the display apparatus for supervisory doctor.

13. The display method for endoscope insertion shape observation apparatus according to claim 11, wherein when the insertion portion is passing through the difficult insertion area of the subject that bends, the image of the subject and the insertion shape of the insertion portion are set to a smaller size than a standard size, and the operation image for operator and the operation information for supervisory doctor are set to a size larger than the standard size to display on the display apparatus for supervisory doctor.

14. The display method for endoscope insertion shape observation apparatus according to claim 11, wherein when the insertion portion forms a loop, the image of the subject, and the operation information for supervisory doctor are set to a smaller size than a standard size and the insertion shape of the insertion portion, and the operation image for operator are set to a larger size than the standard size to display on the display apparatus for supervisory doctor.

15. The display method for endoscope insertion shape observation apparatus according to claim 10, wherein information to display on the display apparatus for supervisory doctor is changed according to the insertion shape or the insertion position.

16. The display method for endoscope insertion shape observation apparatus according to claim 15, wherein:
the operation image for operator includes a left-hand operation image and a right-hand operation image of an operator; and
when the insertion portion is passing through an easy insertion area of the subject that does not bend, the image of the subject, the operation information for supervisory doctor, the insertion shape of the insertion portion and the right-hand operation image of the operator are displayed on the display apparatus for supervisory doctor.

17. The display method for endoscope insertion shape observation apparatus according to claim 15, wherein:
the operation image for operator includes a left-hand operation image and a right-hand operation image of the operator; and
when the insertion portion is passing through a difficult insertion area of the subject that bends, the image of the subject, the operation information for supervisory doctor, the insertion shape of the insertion portion, the right-hand operation image of the operator and the left-hand operation image of the operator are displayed on the display apparatus for supervisory doctor.
